(19) United States
(12) Patent Application Publication
(10) Pub. No.: US 2020/0277651 A1
(43) Pub. Date: Sep. 3, 2020

(54) Nucleic Acid Preparation and Analysis

(71) Applicant: Admera Health LLC, South Plainfield, NJ (US)

(72) Inventors: Jun Huang, South Plainfield, NJ (US); Christopher Kasbek, South Plainfield, NJ (US); Guanghui Hu, South Plainfield, NJ (US); Qingxuan Song, Edison, NJ (US)

(73) Assignee: Admera Health LLC, South Plainfield, NJ (US)

(21) Appl. No.: 15/998,587

(22) PCT Filed: Feb. 16, 2017
(86) PCT No.: PCT/US2017/018052

§ 371 Date: Aug. 16, 2018

Related U.S. Application Data
(60) Provisional application No. 62/296,137, filed on Feb. 17, 2016.

(51) Int. Cl. C12Q 1/6806; C12P 19/34; C12Q 1/6886

(52) U.S. Cl. CPC C12Q 1/6806; C12Q 1/6886; C12P 19/34

(57) ABSTRACT

This invention relates to methods and systems for preparing and analyzing nucleic acids.

### CROSS REFERENCE TO RELATED APPLICATION

This application claims priority to U.S. Provisional Application No. 62/296,137 filed on Feb. 17, 2016. The content of the application is incorporated herein by reference in its entirety.

### FIELD OF THE INVENTION

This invention relates to methods and systems for preparing and analyzing nucleic acids.

### BACKGROUND OF THE INVENTION

Nucleic acid-based detection detects presence of specific nucleic acid (i.e. DNA or RNA) in a test sample. It has been used for various clinical and diagnostic applications. In the case of infectious diseases, nucleic acid-based diagnostics detect DNA or RNA from the infecting organism. For non-infectious diseases, nucleic acid-based diagnostics may be used to detect a specific gene or the expression of a gene associated with disease. A prominent concern confronting clinical and diagnostic applications is the ability to detect clinically significant low-level mutations and minority alleles. The ability to discern mutations is important in many regards, but especially for early cancer detection from tissue biopsies and bodily fluids such as plasma or serum; assessment of residual disease after surgery or radio/chemotherapy; disease staging and molecular profiling for prognosis or tailoring therapy to individual patients; and monitoring of therapy outcome and cancer remission/relapse. Efficient detection of cancer-relevant somatic mutations largely depends on the selectivity of the techniques and methods employed.

The advent of massively parallel DNA sequencing has ushered in a new era of nucleic acid detection by making simultaneous identification of hundreds of billions of base-pairs possible at small fraction of the time and cost of traditional Sanger methods. Because these technologies digitally tabulate the sequence of many individual DNA fragments, unlike conventional techniques which simply report the average genotype of an aggregate collection of molecules, they offer the unique ability to detect minor variants within heterogeneous mixtures. However, such a deep sequencing approach has limitations. Although, in theory, DNA subpopulations of any size should be detectable when deep sequencing a sufficient number of molecules, a practical limit of detection is imposed by errors introduced during sample preparation and sequencing. PCR amplification of heterogeneous mixtures can result in population skewing due to stochastic and non-stochastic amplification biases and lead to over- or under-representation of particular variants (Kanagawa T. Bias and Artifacts in Multitemplate Polymerase Chain Reactions (PCR). J Biosci Bioeng. 2003; 96:317-23.).

Thus, there is a need for systems and methods for enrichment of minority alleles and mutations.

### SUMMARY OF INVENTION

This invention addresses the above-mentioned need by providing systems and methods for enrichment of minority alleles and mutations.

In one aspect, the invention provides a method for exponential amplification of one or more double-stranded target nucleic acid molecules. The method includes (a) ligating to each double-stranded target nucleic acid molecule an adapter to produce an end-linked double-stranded nucleic acid molecule, said adapter comprising (i) a paired region and (ii) an unpaired region; (b) providing (i) an adapter primer that is complementary to, or hybridizes to, a primer binding site in the complement of the unpaired region and (ii) a target-specific primer that is complementary to, or hybridizes to, a binding site in the target nucleic acid molecule; and (c) amplifying the end-linked double-stranded nucleic acid molecule in an amplification reaction comprising the adapter primer and the target-specific primer to produce a first amplified molecule.

The unpaired region can be a loop, a 5′ and/or 3′ overhang, or a bubble. In some embodiments, the unpaired region is a loop. In that case, the loop can contain a uracil and the method can comprise cleaving the loop by uracil DNA glycosylase (UDG) before the amplifying step. The target-specific primer, the adapter primer, or both can each contain a tag sequence at the 5′ end. The tag sequence can be used to facilitate detection, sequencing, cloning and/or amplification as described below.

In other embodiments, the amplification reaction comprises a first blocker comprising a first sequence that (i) is matched or complementary to the wild-type allele in the target nucleic acid molecule and (ii) is capable of being extended by a DNA polymerase. The amplification reaction can further comprise a second blocker having a second sequence that is matched or complementary to the complement of the wild-type allele. The first or second blocker contains one or more modified nucleic acids or linkages. For example, the first blocker or the second blocker can have a modified nucleic acid or linkage at the 3′ end. Examples of the modified nucleotides or linkages include PNA, LNA, a 2′-O-Methyl nucleic acid, a 2′-O-Alkyl nucleic acid, a 2′-fluoro nucleic acid, a phosphorothioate linkage, and any combination thereof. In some embodiments, the first or second blocker does not overlap with either the adaptor primer or the target-specific primer.

In the above-described method, the target nucleic acid molecule can be a cell free nucleic acids (cfNA), cell free DNA (cfDNA) or circulating tumor DNA (ctDNA). The target nucleic acid molecule can be 20 bp-20 kb in length, such as 20 bp-2 kb, 20 bp-1 kb, or 20 bp-200 bp in length. In some examples, the target nucleic acid molecule spans a region encoding EGFR T790M, EGFR L858R, BRAF V600E, BRAF V600K, BRAF V600D, BRAF V600G, BRAF V600A, or BRAF V600R.

The above-described method can be used in a method for obtaining the sequence of one or more double-stranded target nucleic acid molecules. This method includes obtaining a first amplified molecule produced according to the above-described method, amplifying the first amplified molecule in a second amplification reaction comprising a pair of primers, each primer having a barcode sequence, to generate a set of second amplified molecules, and sequencing the second amplified molecules.

The above-described method can be used in a method for evaluating a subject having cancer or suspected of having cancer. This evaluation method includes obtaining a biological sample from the subject; and performing an assay to determine the presence or absence of one or more target nucleic acid molecules in the biological sample according to the method described above. Examples of the biological sample include serum, plasma, whole blood, saliva, and sputum. In some embodiments, the evaluation method can also include determining or recommending a treatment course of action based on the presence of said one or more target nucleic acid molecules. In others, the method further comprises a step of administering said treatment when said one or more target nucleic acid molecules are present. The amplification reaction can be a multiplex amplification reaction.

In a second aspect, the invention provides a kit for amplification of a target nucleic acid molecule. The kit contains (a) an adapter comprising (i) a paired region and (ii) an unpaired region; (b) an adapter primer that is complementary to a primer binding site in the complement of the unpaired region, and (c) a target-specific primer that is complementary to a binding site in the target nucleic acid molecule. The kit can further include (d) a first blocker comprising a first sequence that (i) is matched or complementary to the wild-type allele in the target nucleic acid molecule and (ii) is capable of being extended by a DNA polymerase, or (e) a second blocker having a second sequence that is matched or complementary to the complement of the wild-type allele. The unpaired region can be a loop, a 5′ and/or 3′ overhang, or a bubble. Preferably, the unpaired region is a loop. The loop can contain a uracil. At least one of the primers and blockers contains one or more modified nucleic acids or modified linkages, including but not limited to PNA, LNA, a 2′-O-Methyl nucleic acid, a 2′-O-Alkyl nucleic acid, a 2′-fluoro nucleic acid, a phosphorothioate linkage, and any combination thereof. In some embodiments, the target-specific primer or the adapter primer or both can each contain a tag sequence at the 5′ end. The tag sequence can be used to facilitate detection, sequencing, cloning and/or amplification. For example, the tag can include a sequence that is identical or substantially identical to a known sequencing primer so that its complement generated during a nucleic acid amplification/extension event can provide a binding site for the sequencing primer. Examples of the sequencing primer include an Illumina P5 barcode primer, an Illumina P7 barcode primer, an Ion Torrent A barcode primer, and an Ion Torrent P1 barcode primer. In that case, the kit can also include one or more of these tag-containing primers and known sequencing primers, such as Illumina P5 barcode primer, an Illumina P7 barcode primer, an Ion Torrent A barcode primer, and an Ion Torrent P1 barcode primer.

The details of one or more embodiments of the invention are set forth in the description below. Other features, objectives, and advantages of the invention will be apparent from the description and from the claims.

### BRIEF DESCRIPTION OF THE DRAWINGS

FIG. 1 is a diagram showing schematics of an exemplary method of amplifying gDNA fragments. (TCGNNNNNN, SEQ ID NO: 1; NNNNNNCGAT, SEQ ID NO: 2)

FIGS. 2A, 2B, and 2C are diagrams showing an exemplary system of this invention: FIG. 2A: A blocker anneals to a wild type allele and the extended blocker blocks PCR amplification of the outer primers. FIG. 2B: Each blocker oligo has a mismatch at 3′ end and does not stay annealed to mutant allele, thus allows amplification by outer primers. FIG. 2C: Non-specific extension only results in amplification failure of the particular template allele in this singular cycle. No false positive PCR product is formed.

### DETAILED DESCRIPTION OF THE INVENTION

This invention relates to methods and systems for preparing and analyzing nucleic acids. The methods and systems provided herein are useful for detecting specific nucleic acids and for preparing nucleic acids for analysis (e.g., for sequencing).

### Approach and Systems

This invention is based, at least in part, on a novel approach to enrich DNA to efficiently and accurately amplify targeted regions. In one aspect, the invention provides a new way to incorporate a unique identifier (UID) to amplification from a specific target. Comparing to conventional PCR-based method, the approach of this invention allows one to obtain more accurate sequencing, thus higher ability to detect rare mutations. This is particularly useful in detecting and quantifying a minor DNA population (e.g., ctDNA or cfDNA) in a large DNA population as one can use UID to identify the quantity of the minor DNA templates that are from the enrichment products.

In some aspects of the approach disclosed herein methods are provided of preparing one or more nucleic acids for analysis that involve:

(a) contacting each double-stranded target nucleic acid molecule with an adapter under condition for nucleic acid ligation to produce an end-linked double-stranded nucleic acid molecule, said adapter comprising (i) a paired region and (ii) an unpaired region;

(b) providing (i) an adapter primer that is complementary to a primer binding site in the complement of the unpaired region and (ii) a target-specific primer that is complementary to a binding site in the target nucleic acid molecule; and

(c) amplifying the end-linked double-stranded nucleic acid molecule in an amplification reaction comprising the adapter primer and the target-specific primer to produce a first amplified molecule.

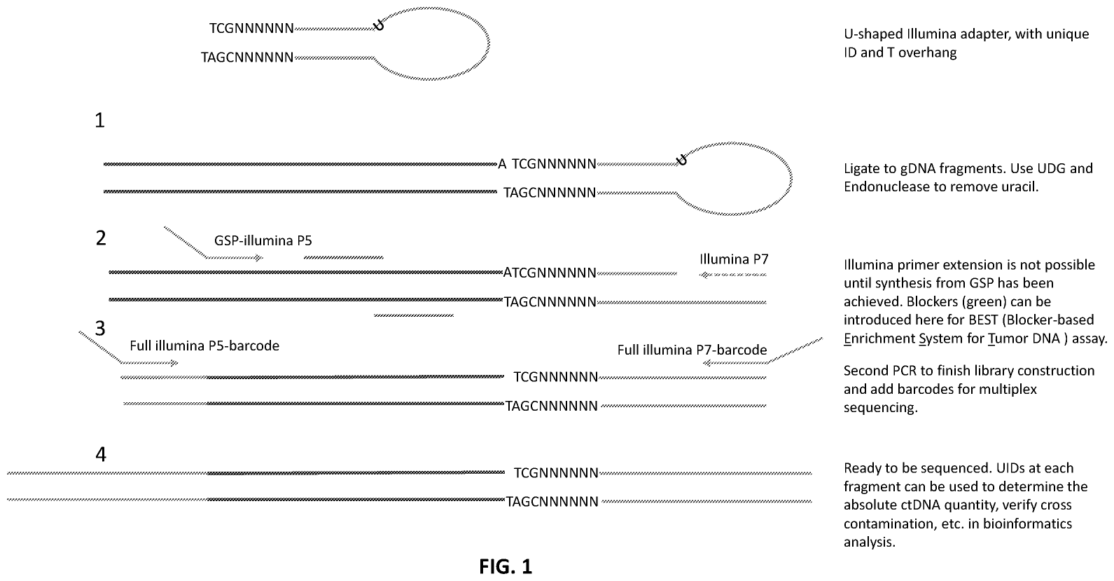

For example, FIG. 1 presents schematics of an exemplary method of amplifying gDNA fragments. Shown at the top of the figure is a U-shaped Illumina adapter, with unique ID and T overhang. Although a U-shaped Illumina adapter is used here as an example, many other adapters (e.g., Y adapters, bubble adapters, and Splinkerette adapters) can also be used as described below. Such an adapter does not have a primer-binding site in a target nucleic acid of interest to initiate PCR.

As shown in step 1 in FIG. 1, this adapter is ligated to the target gDNA fragments. In this particular example, the loop region of the adapter has a uracil (U). Once the adapter is ligated, one can use Uracil DNA glycosylase (UDG) or/and other suitable endonuclease to remove uracil or cut open the loop region, thereby generating a 5′ staggered end/overhang.

As shown in step 2 in FIG. 1, this 5′ overhang has a sequence identical or substantial identical to an adapter primer (e.g., an Illumina P7 primer) so that the complement of the overhang sequence provides a primer binding site for the adapter primer, which can be used in combination with a specific site within the target gDNA for amplification reaction.

In step 2, the gDNA molecules are contacted by a target-specific primer (or gene specific primer, GSP) and the adapter primer (e.g., an Illumina P7 primer). At beginning, nucleic acid synthesis/extension from the Illumina P7 primer is not possible until nucleic acid synthesis from the gene specific primer has been achieved. During this synthesis/extension process, blockers (shown as two bars in FIG. 1) can be introduced. It's only after the gene-specific primer (e.g., GSP-illumina P5) extends to the adapter region that the adapter primer can anneal and start amplification. This design is important to avoid massive non-specific amplification.

The primer-extension products from step 2 can be used as a target-specific template to obtain additional copies. Multiple different gene-specific primers can be used similarly but in a parallel, multiplex manner to construct libraries for further high throughput analysis. In some embodiments, a nested target specific primer (nested with respect to the target specific primer of step 2) can be used too.

The target-specific primer(s) hybridizes to a portion of the target nucleic acid. In some embodiments, pools of different target-specific primers can be used that hybridize to different portions of a target nucleic acid. In some embodiments, use of different target specific primers can be advantageous because it allows for generation of different extension products having overlapping but staggered sequences relative to a target nucleic acid. In some embodiments, different extension products can be sequenced to produce overlapping sequence reads. In some embodiments, overlapping sequence reads can be evaluated to assess accuracy of sequence information, fidelity of nucleic acid amplification, and/or to increase confidence in detecting mutations, such as detecting locations of chromosomal rearrangements (e.g., fusion breakpoints). In some embodiments, pools of different target-specific primers can be used that hybridize to different portions of different target nucleic acids present in sample. In some embodiments, use of pools of different target-specific primers is advantageous because it facilitates processing (e.g., amplification) and analysis of different target nucleic acids in parallel. In some embodiments, up to 2, up to 3, up to 4, up to 5, up to 6, up to 7, up to 8, up to 9, up to 10, up to 15, up to 20, up to 100 or more pools of different first target-specific primers are used. In some embodiments, 2 to 5, 2 to 10, 5 to 10, 5 to 15, 10 to 15, 10 to 20, 10 to 100, 50 to 100, or more pools of different first target-specific primers are used.

In some embodiments, the target specific primer may comprise an additional sequence 5′ to the hybridization sequence. This additional sequence may include barcode, index, adapter sequences, or sequencing primer sites. For example, a second PCR is carried out as shown in step 3 of FIG. 1 to finish library construction and add barcodes (illumina P5 and illumina P7 barcodes) for multiplex sequencing. In some embodiments, amplified products are purified after step 2, 3, or 4. The amplification products from the second PCR (step 4 FIG. 1) are ready for analysis. For example, the products at step 4 can be sequenced (e.g., using next generation sequencing platform).

UIDs at each fragment can be used to determine the absolute ctDNA quantity, verify cross contamination, and carry out other desired analysis, e.g., bioinformatics analysis. UID is a commonly used technique to identify PCR/sequencing errors. Normally UID is tagged to each DNA fragment, and fragments of interest are fished out using probes (e.g., www.genomics.agilent.com/article.jsp?pageId=3083). However, this probe-based enriching approach is costly and time-consuming. In contrast, retrieving fragments of interest using the adapter and gene-specific primer described herein shortens the library prep from about 4-7 days to about 1 day. Additional advantages of this method as compared to conventional PCR method include more accurate sequencing, thus higher ability to detect rare mutations, and accurate quantification of a rare DNA population (e.g., ctDNA) in total DNA population (e.g., cfDNA) because one can use UID to identify how many tumor DNA templates the enrichment product is from.

### Adapters

The present invention utilizes oligonucleotide adapters for the exponential amplification of a nucleic acid sequence wherein the resulting amplified product will have a different nucleic acid sequence on each end.

The adapter comprises a ligatable end and at least one unpaired or single-stranded region. The unpaired region can be of any appropriate size, for example, from at least about 3-200 nucleotides (nt.) such as 5-150 nt., 10-100 nt., and 15-50 nt. In one embodiment, the length of the unpaired region is sufficient to permit primer binding for amplification, wherein at least the 3′ region of the primer can match to the unpaired region of the adapter. A single-stranded region, tail, or overhang, is a single-stranded nucleic acid sequence extension at either end (e.g., 5′ end; 3′ end) of an adapter, in which the longer strand of the adapter is not base paired with a reverse complementary sequence in the other (opposite) strand (see, e.g., FIG. 1), as will be understood by one of skill in the art. In one embodiment, the overhang is at least about 3-200 nucleotides (nt.) in length, such as 5-150 nt., 10-100 nt., and 15-50 nt.

Examples of adapter can be used to practice the invention here include double-stranded adapter, U-shaped adapter as shown in FIG. 1, Y-adapters and bubble adapter as described in US 20100222238, and splinkerette type adapter as described in Uren et al., Nat Protoc. 2009; 4(5):789-98.

An adapter can comprise at least one blocking group. As used herein, a blocking group is an agent or substituent that prevents nucleic acid sequence extension (e.g., by DNA polymerase or DNA ligase) and hence also prevents amplification of a nucleic acid sequence comprising the blocking group. Examples of 3′ blocking groups which may be present on a terminal 2′ deoxynucleotide include 3′ deoxy, 3′ phosphate, 3′ amino, or 3′-O—R nucleotide where R represents an alkyl, allyl, aryl or heterocyclic substituent. In a particular embodiment, the second asymmetrical tail adapter comprises a blocking group. As used herein, “double stranded” refers to a paired nucleic acid sequence, wherein the two strands are substantially complementary to each other such that the two strands can form a paired structure (e.g., a double helix). As will be understood by the person of skill in the art, the two strands may contain one or more mismatches and still retain a paired structure. In a particular embodiment, the paired structure is stable.

As described herein, an adapter can comprise a ligatable end. A ligatable end is a sequence in a double-stranded oligonucleotide that has either a blunt end or a sticky-end. As will be understood by one of skill in the art, a blunt end has no 5′ or 3′ overhang in a double stranded nucleic acid molecule and a sticky end has either a 5′ or a 3′ overhang. Both blunt ends and sticky ends can be ligated to another compatible end. As used herein, a compatible end is a blunt end that can ligate with another blunt-ended nucleic acid sequence, or a sticky end comprising an overhang which can ligate with another sticky end that comprises essentially the reverse complementary overhang. Thus, sticky ends permit sequence-dependent ligation, whereas blunt ends permit sequence-independent ligation. Compatible ends and, thus, ligatable ends can be produced by any known methods that are standard in the art. For example, compatible ends of a nucleic acid sequence are produced by restriction endonuclease digestion of the 5′ and/or 3′ end. In another embodiment, compatible ends of a nucleic acid sequence are produced by introducing (for example, by annealing, ligating, or recombining) an adapter to the 5′ end and/or 3′ end of the nucleic acid sequence, wherein the adapter comprises a compatible end, or alternatively, the adapter comprises a recognition site for a restriction endonuclease that produces a compatible end on cleavage. Blunt ends can be produced by digestion with a site-specific endonuclease (e.g., a restriction endonuclease), a non-specific double-stranded DNA specific endonuclease (e.g., DNA polymerase I in the presence of Mn²⁺) or by random shearing (e.g., by sonication, acoustic energy, or hydrodynamic shearing by forcing a DNA solution through a small orifice under pressure). After random shearing or DNAase digestion the DNA ends are often frayed (contain short 5′ or 3′ overhangs with or without terminal phosphate groups). The frayed ends are converted to ligatable ends by blunt-ending, or healing, using one or more of the following: a DNA polymerase, a mixture of dATP, dCTP, dGTP and dTTP, a DNA polymerase having strong 3′ to 5′ and 5′ to 3′ exonuclease activities, polynucleotide kinase, ATP, a single stranded DNA specific exonuclease, a single stranded DNA specific endonuclease.

As described herein, the above adapter is ligated to a target nucleic acid so that a primer-binding site for an adapter primer can be introduced. As shown in FIG. 1, this adapter primer and a gene-specific primer together allow primer extension and/or amplification of the target nucleic acid.

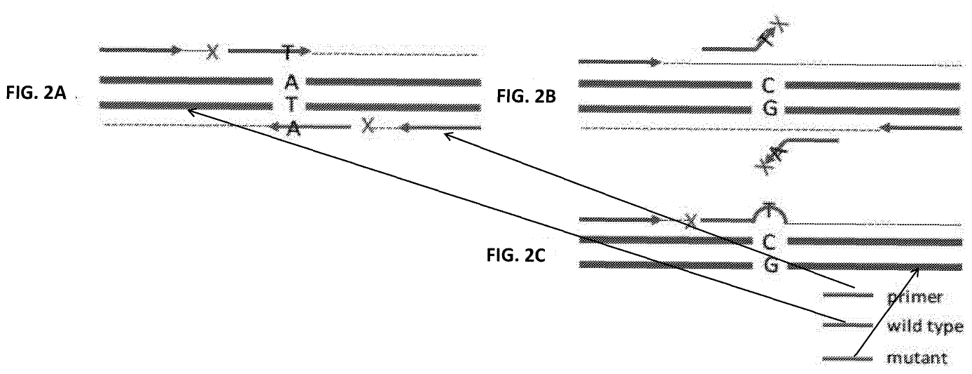

As used herein, a primer binding site comprises a sequence that binds a whole primer length, or the primer binding site can comprise a sequence that binds to a sufficient portion of the 3′ end of the primer, wherein the portion is sufficient to permit primer binding, e.g., for primer extension and/or amplification. In preferred embodiments, the unpaired/single-stranded region of the adapter does not directly provide or otherwise comprise a binding site for the adapter primer. Rather, the binding site is generated only if the primer extension from the gene-specific primer has been achieved and filled in the staggered end. See step 3 of FIG. 1. In other words, the adapter primer (e.g., Illumina P7 as shown in FIG. 2) hybridizes to the complement of the unpaired single-stranded region of the adapter.

In certain embodiments, methods provided herein include using oligonucleotide blockers that are matched to or complementary to a particular nucleic acid variant (such as a wild type variant). Examples of such blockers include those described in PCT/US2016/057805 and U.S. Application No. 62/244,279, the content of which is incorporated by reference in its entirety. These blockers can block or suppress the amplification of that particular nucleic acid variant, thereby allowing enrichment of other variants (e.g., mutant variants). Accordingly, the present invention provides enrichment reaction systems and methods for detecting the presence or absence of a nucleic acid variant in a target region.

In these embodiments, an enrichment reaction system of this invention comprises the above-described primers, blockers, and essential ingredients for PCR amplification. As shown in FIGS. 2A-C, the system of this invention can have (i) a first blocker that binds or hybridizes to the same strand or sequence as the forward primer and (ii) a second blocker and a reverse primer that bind or hybridize to the opposite strand and/or complementary sequence. A primer pair (i.e., a forward primer and a reverse primer) is used to amplify the region containing hotspot mutation and blockers are used to block the amplification of a nucleic acid variant (e.g., an abundant allelic variant such as a wild type allele). A blocker is an oligo complementary to the nucleic acid variant (e.g., wild type allele). Its 3′ end is designed to match perfectly to that variant of interest. For example, it perfectly anneals to the wild type allele and is able to be extended by a DNA polymerase. Melting temperature is highly correlated with the length of an oligo. By extending the length of the blocker oligo, the blocker withstands a higher reaction temperature and thus stays associated with the wild type allele. On the other hand, at an initial low reaction temperature, the blocker can also anneal to mutant allele; however, because the mutated bases of the mutant allele do not match the 3′ end of the blocker, extension does not occur. This results in dissociation of the blocker from mutant allele once temperature rises. Therefore, the blocker is tightly annealed to wild type while is easily denatured from mutant. Primers are in the reaction to amplify the region of interest. Since the blocking oligo is annealed to wild type, the primer extension cannot continue pass the blocked region, and thus mutant allele is preferentially amplified.

For example, as shown in FIG. 2A, the blocker anneals to a wild type allele and the extended oligo blocker blocks PCR amplification of the outer primers. In FIG. 2B, each oligo blocker has a mismatch at 3′ end and does not stay annealed to mutant allele, thus allows amplification by outer primers. In FIG. 2C, a non-specific extension only results in amplification failure of the particular template allele in this singular cycle. No false positive PCR product is formed.

The system disclosed herein is superior to allele-specific PCR (AS-PCR), also known as amplification mutation refractory system (Newton C, et al. Nucleic acids. 1989; 17(7):2503-2516), which is a tried-and-true technique to enrich hotspot mutations. In the AS-PCR approach, the 3′ end of the primers is designed to match perfectly to a variant of interest and allow specific mutant amplification. Qiagen Therascreen EGFR and Roche Cobas EGFR systems, for instance, adopted this technique. However, the inherent disadvantage of non-specific extension of the allele-specific primer lowers sensitivity that leads to unreliable discrimination between rare somatic mutant and wild type. LOD is documented 0.5%-7.02% for Therascreen and 5% for Cobas.

### Blocker

As mentioned above, a blocker (herein sometimes referred to as “blocking oligo”) is complementary to a particular nucleic acid variant to be suppressed, such as abundant allelic variant (e.g., a wild type allele). Such a blocker may be designed as short oligomers that are single-stranded and have a length of 100 nucleotides or less, more preferably 50 nucleotides or less, still more preferably 30 nucleotides or less and most preferably 20 nucleotides or less with a lower limit being approximately 5 nucleotides.

The blocker, as well as primers disclosed herein, can in some cases be modified by a variety of methods known in the art to protect against 3′ or 5′ exonuclease activity. The blocker can include one or more modifications to protect against 3′ or 5′ exonuclease activity and such modifications can include but are not limited to 2′-O-methyl ribonucleotide modifications, phosphorothioate backbone modifications, phosphorodithioate backbone modifications, phosphoramidate backbone modifications, methylphosphonate backbone modifications, 3′ terminal phosphate modifications and 3′ alkyl substitutions. In some embodiments, the blocker is resistant to 3′ and/or 5′ exonuclease activity due to the presence of one or more modifications.

Its 3′ end is designed to match perfectly to a particular nucleic acid variant of interest. In some embodiments, a blocker perfectly anneals to the wile type allele and is able to be extended by a DNA polymerase.

Melting temperature (Tm) of the blocker is highly correlated with its length. The Tm of the blocker can range from 40° C. to 70° C., such as 40° C. to 70° C., 41° C. to 69° C., 42° C. to 68° C., 43° C. to 67° C., 44° C. to 66° C., or about 53° C. to about 56° C., or any range in between. In yet other embodiments, the Tm of the blocker can be about 3° C. to 6° C. higher than the anneal/extend temperature in the PCR cycling conditions employed during amplification.

In some embodiments, the blocker is not cleaved during PCR amplification. According to the present invention, the blocker can be either extendable or non-extendable. In some embodiments, the blocker can comprise a non-extendable blocker moiety at its 3′-end. In some embodiments, the blocker can further comprise other moieties (including, but not limited to additional non-extendable blocker moieties, quencher moieties, fluorescent moieties, etc.) at its 3′-end, 5′-end, and/or any internal position in between. In others, the blocker is extendable and does not contain any non-extendable blocker moiety at its 3′-end. In that case, the blocker is extended during PCR. By extending the length of the blocking oligo, the blocker withstands a higher reaction temperature and thus stays associated with the wild type allele.

### Primers

According to the present invention, a forward primer and/or reverse primer can be designed to be complementary (fully or partially) to various suitable positions relative to one or more nucleic acid variants of interest. For example, the 3′ region of the forward primer or reverse primer when hybridized to the target region in some cases can be located 0, 5, 10, 15, 20, 25, 30, 35, 40, 45, 50, 55, 60, 80, 100, 250, 500, 1000, 2000 or more nucleotides away from one or more nucleic acid variants in the target region. In some embodiments, the 3′ region of the forward primer or reverse primer when hybridized to the target region is located less than about 30 nucleotides away from one or more nucleic acid variants in the target region. The primers can be oligomers ranging from about 10-50, e.g., about 15-30, about 16-28, about 17-26, about 18-24, or about 20-22, or any range in between, nucleotides in length.

In some instances, the forward or reverse primer and blockers can overlap and compete for hybridizing to a partial or full target region. For example, the primer and blockers can overlap by 0, 5, 10, 15, or more nucleotides. In some embodiments, the primer and blockers do not overlap or compete for hybridizing to a partial or full target region at all.

The above-discussed primers and/or the blockers can comprise one or more modified nucleobases or nucleosidic bases different from the naturally occurring bases (i.e., adenine, cytosine, guanine, thymine and uracil). In some embodiments, the modified bases are still able to effectively hybridize to nucleic acid units that contain adenine, guanine, cytosine, uracil or thymine moieties. In some embodiments, the modified base(s) may increase the difference in the Tm between matched and mismatched target sequences and/or decrease mismatch priming efficiency, thereby improving not only assay specificity, bust also selectivity.

Modified bases are considered to be those that differ from the naturally-occurring bases by addition or deletion of one or more functional groups, differences in the heterocyclic ring structure (i.e., substitution of carbon for a heteroatom, or vice versa), and/or attachment of one or more linker arm structures to the base. In some embodiments, all tautomeric forms of naturally occurring bases, modified bases and base analogues may also be included in the oligonucleotide primers and blockers of the invention.

Some examples of modified base(s) may include, for example, the general class of base analogues 7-deazapurines and their derivatives and pyrazolopyrimidines and their derivatives (see e.g., WO 90/14353 and US20100285478, the content which are incorporated herein by reference in their entireties). Examples of base analogues of this type include, for example, the guanine analogue 6-amino-1H-pyrazolo[3,4-d]pyrimidin-4(5H)-one (ppG), the adenine analogue 4-amino-1H-pyrazolo[3,4-d]pyrimidine (ppA), and the xanthine analogue 1H-pyrazolo[4,4-d]pyrimidin-4(5H)-6(7H)-dione (ppX). These base analogues, when present in an oligonucleotide of some embodiments of this invention, strengthen hybridization and can improve mismatch discrimination.

Additionally, in some embodiments, modified sugars or sugar analogues can be present in one or more of the nucleotide subunits of an oligonucleotide in accordance with the invention. Sugar modifications include, but are not limited to, attachment of substituents to the 2′, 3′ and/or 4′ carbon atom of the sugar, different epimeric forms of the sugar, differences in the α or β-configuration of the glycosidic bond, and other anomeric changes. Sugar moieties include, but are not limited to, pentose, deoxypentose, hexose, deoxyhexose, ribose, deoxyribose, glucose, arabinose, pentofuranose, xylose, lyxose, and cyclopentyl.

Locked nucleic acid (LNA)-type modifications, for example, typically involve alterations to the pentose sugar of ribo- and deoxyribonucleotides that constrains, or “locks,” the sugar in the N-type conformation seen in A-form DNA. In some embodiments, this lock can be achieved via a 2′-O, 4′-C methylene linkage in 1,2:5,6-di-O-isopropylene-α-D-allofuranose. In other embodiments, this alteration then serves as the foundation for synthesizing locked nucleotide phosphoramidite monomers. (See, for example, Wengel J., Ace. Chem. Res., 32:301-310 (1998), U.S. Pat. No. 7,060,809; Obika, et al., Tetrahedron Lett 39: 5401-5405 (1998); Singh, et al., Chem Commun 4:455-456 (1998); Koshkin, et al., Tetrahedron 54: 3607-3630 (1998), the disclosures of each of which are incorporated herein by reference in their entireties.)

In some preferred embodiments, the modified bases include 8-Aza-7-deaza-dA (ppA), 8-Aza-7-deaza-dG (ppG), 2′-Deoxypseudoisocytidine (iso dC), 5-fluoro-2′-deoxyuridine (fdU), LNA, or 2′-0,4′-C-ethylene bridged nucleic acid (ENA) bases. Other examples of modified bases that can be used in the invention are described in U.S. Pat. No. 7,517,978, the disclosure of which is incorporated herein by reference in its entirety.

Many modified bases, including for example, LNA, ppA, ppG, 5-Fluoro-dU (fdU), are commercially available and can be used in oligonucleotide synthesis methods well known in the art. In some embodiments, synthesis of modified primers and blockers can be carried out using standard chemical means also well known in the art. For example, in certain embodiments, the modified moiety or base can be introduced by use of a (a) modified nucleoside as a DNA synthesis support, (b) modified nucleoside as a phosphoramidite, (c) reagent during DNA synthesis (e.g., benzylamine treatment of a convertible amidite when incorporated into a DNA sequence), or (d) by post-synthetic modification.

Due to the flexibility of the nucleotide structure, some mismatched base pairs can partially conform for Watson-Crick binding. This feature renders the blocker inefficient as it can also be extended on mutant allele. LNA and some other nucleic acid analogues with a more rigid structure can be used to alleviate the problem. The blockers used can contain one or two LNA at and/or near the variant of interest.

In some embodiments, the primers or blockers are synthesized so that the modified bases are positioned at the 3′ end. In some embodiments, the modified base is located between, 1-6 nucleotides, e.g., 2, 3, 4 or 5 nucleotides away from the 3′-end of the primer or blocker. In some preferred embodiments, the primers or blockers are synthesized so that the modified bases are positioned at the 3′-most end.

Modified inter-nucleotide linkages can also be present in primers and blockers disclosed in this invention. Such modified linkages include, but are not limited to, peptide, phosphate, phosphodiester, alkylphosphate, alkanephosphonate, thiophosphate, phosphorothioate, phosphorodithioate, methylphosphonate, phosphoramidate, substituted phosphoramidate and the like. Several further modifications of bases, sugars and/or inter-nucleotide linkages, that are compatible with their use in oligonucleotides serving as blockers and/or primers, will be apparent to those of skill in the art.

DNA polymerase with 3′ to 5′ exonuclease activity is able to remove mismatched bases from 3′ end of an oligo, and thus the discriminating bases from the blockers can be removed. A phosphorothioate bond is more resistant to exonuclease activity than the indigenous phosphodiester bond; therefore, it is used at the 3′ of the blockers.

### Methods

The above-described system can be used in various methods for identifying, enriching, and/or quantifying a target nucleic acid or an allele variant in a sample.

A method disclosed in the invention generally includes amplifying a target region with a forward primer and a reverse primer in the presence of one or more blockers. The blocker includes a sequence complementary to the target region in the absence of the nucleic acid variant to be enriched. The methods can further include detecting amplification of the target region. The methods of the present invention allow one to detect nucleic acid variants with very high sensitivity, in some cases at a limit of detection (LOD) of about 0.01% to 0.001%.

As shown in FIG. 2A, when a blocker anneals to a variant (e.g., a wild type variant), the blocker extends and blocks PCR amplification, thereby preventing the extension of a distant forward primer or reverse primer. In some embodiments, the forward or reverse primer can be located 0, 5, 10, 15, 20, 25, 30, 35, 40, 45, 50, 55, 60, 80, 100, 250, 500, 1000, 2000 or more nucleotides away from the region where the blocker hybridizes. In some embodiments, the blocker has a sufficiently high Tm that it is not displaced by a replicating forward primer or reverse primer. In some embodiments, the enzyme used during the amplification reaction does not comprise a strand displacement activity.

Various DNA polymerases can be used in this invention. In some cases, the enzymes employed with the methods of the present invention for amplification of the target region include but are not limited to high-fidelity DNA polymerases and repair enzymes that possess 3′ exonuclease repair activity. Exemplary enzymes for use with the methods of the invention can include but are not limited to, Pfu Turbo Hotstart DNA Polymerase, Phusion™ Hot Start High Fidelity DNA Polymerase, Phusion Hot™ Start II High Fidelity DNA Polymerase, Phire™ Hot Start DNA Polymerase, Phire™ Hot Start II DNA Polymerase, KOD Hot Start DNA Polymerase, Q5 High Fidelity Hot Start DNA Polymerase, AmpliTaq, Phusion HS II, Deep Vent, and Kapa HiFi DNA polymerase.

General methods for amplifying nucleic acid sequences are well known in the art. Any such methods can be employed with the methods of the present invention. In some embodiments, the amplification uses digital PCR methods, such as those described, for example, in Vogelstein and Kinzler (“Digital PCR,” PNAS, 96:9236-9241 (1999); incorporated by reference herein in its entirety). Such methods include diluting the sample containing the target region prior to amplification of the target region. Dilution can include dilution into conventional plates, multiwell plates, nanowells, as well as dilution onto micropads or as microdroplets. (See, e.g., Beer N R, et al., “On-chip, real-time, single-copy polymerase chain reaction in picoliter droplets,” Anal. Chem. 79(22):8471-8475 (2007); Vogelstein and Kinzler, “Digital PCR,” PNAS, 96:9236-9241 (1999); and Pohl and Shih, “Principle and applications of digital PCR,” Expert Review of Molecular Diagnostics, 4(1):41-47 (2004); all of which are incorporated by reference herein in their entirety.). When combined with digital PCR, the present invention can greatly increase the sensitivity of digital PCR. This is due in part to the fact that the current invention provides methods for significantly suppressing (e.g., at least about 60%, 65%, 70%, 75%, 80%, 85%, 90%, 95%, 97%, 98%, 99%, 99.9%, 99.99%, 99.999%, or about 100%) wild-type associated background, when interrogating genetic events, including for example rare genetic events. The sensitivity of targeting provided by the methods of the present invention allows far higher target loading in the individual volume elements of the single digital PCR reactions.

When running PCR (e.g., digital PCR) for detecting rare genetic events, most of the events present in a given reaction mixture will be of a wild-type sequence while very few will contain the rare genetic event. The methods of the present invention provide for very effective wild-type suppression, for example greater than 1:10,000 as described herein. In some embodiments, 10,000 wild-type targets can be present in each PCR digital element while still allowing for detection of a single rare target due to the effective suppression of the wild-type amplification combined with not suppressing amplification of the single rare target.

The methods of the present invention can further include detecting amplification of the target region using any detection method well known in the art. For example, detection can be by obtaining melting curves for the amplified products, by mass spectrometry, or by sequencing of the amplified products. Amplification products will exhibit different melting curves depending on the type and number of nucleic acid variants in the amplification product. Methods for determining melting curves have been well described and are well known to those of skill in the art and any such methods for determining melting curves can be employed with the methods of the present invention. Methods for the use of mass spectrometry as well as methods for sequencing nucleic acids are also all well known in the art. See, for e.g., Sambrook and Russell, Molecular Cloning: A Laboratory Manual, (3 ed.) (2001) and Plum, Optical Methods, Current Protocols in Nucleic Acid Chemistry, 2001-2011); all of which are incorporated by reference herein in their entirety). See, for e.g., Current Protocols in Nucleic Acid Chemistry, 2001-2011, specifically Liquid Chromatography-Mass Spectrometry Analysis of DNA Polymerase Reaction Products; incorporated by reference herein in its entirety. Methods for nucleic acid sequencing are also routine and well known by those skilled in the art and any methods for sequencing can be employed with the methods of the present invention. See, e.g., Current Protocols in Molecular Biology, 1995-2010; incorporated by reference herein in its entirety.

The methods of the invention further include detecting amplification of the target region by comparing the quantity of the amplified product to a predetermined level associated with the presence or absence of the nucleic acid variant in the target region. Methods for detecting amplification or determining the quantity of an amplified product are well known in the art and any such methods can be employed. See, e.g., Sambrook and Russell, Molecular Cloning: A Laboratory Manual (3^{rd} ed.) (2001) and Gallagher, Current Protocols Essential Laboratory Techniques, 2008); all of which are incorporated by reference herein in their entirety.

The nucleic acid variants that can be detected by the methods of the present invention include mutations in the target region, deletions in the target region, and/or insertions in the target region. Deletions include removal of a nucleotide base from the target region. Deletions that can be detected include deletion of 1, 2, 3, 4, 5 or more (such as hundreds of or thousands of) nucleotide bases from the target region. Mutations can include but are not limited to substitutions (such as transversions and transitions), abasic sites, crosslinked sites, and chemically altered or modified bases. Mutations that can be detected include mutation of 1, 2, 3, 4, 5 or more nucleotide bases within the target region. Insertions include the addition of a nucleotide into a target region. Insertions that can be detected can include insertion of 1, 2, 3, 4, 5 or more (such as hundreds of or thousands) nucleotide bases into the target region. In some embodiments, a deletion, a mutation and/or an insertion is detected by the methods of the present invention.

The system and method disclosed herein minimize false positives, the biggest weakness of AS-PCR. Instead of extending mutant DNA, the blocking oligos anneal to the wild type DNA and block its amplification and, consequently, the mutant DNA is preferentially amplified (FIGS. 2A and 2B). The occasional allelic non-specific blocker extension has nearly no effect on enrichment outcome (FIG. 2C). Amplification of the residual non-blocked wild type can be recognized in subsequent NGS sequencing.

As disclosed herein, the system and method described in this invention efficiently block more than e.g., 99.9% of wild type amplification and result in the significantly augmented presence of a mutant allele from one in ten thousand up to one in seven that is from about 0.01% to about 14%. Rare false positives are not introduced by the intrinsic non-specific extension of allele-specific oligos but rather only result from the nucleotide incorporation errors by DNA polymerase. Using high-fidelity polymerase with 3′ to 5′ exonuclease activity further reduces the rare false positives.

### Uses and Applications

The method disclosed herein can be used for enriching or detecting various target nucleic acid of interest. The target nucleic acid can be a part of a double stranded nucleic acid or a single-stranded nucleic acid.

Sources of nucleic acid samples that can be used include, but are not limited to, human cells such as circulating blood, cultured cells and tumor cells. Also other mammalian tissue, blood and cultured cells are suitable sources of template nucleic acids. In addition, viruses, bacteriophage, bacteria, fungi and other micro-organisms can be the source of nucleic acid for analysis. The DNA may be genomic or it may be cloned in plasmids, bacteriophage, bacterial artificial chromosomes (BACs), yeast artificial chromosomes (YACs) or other vectors. RNA may be isolated directly from the relevant cells or it may be produced by in vitro priming from a suitable RNA promoter or by in vitro transcription. The present invention may be used for the detection of variation in genomic DNA whether human, animal or other. It finds particular use in the analysis of inherited or acquired diseases or disorders. A particular use is in the detection of inherited diseases and cancer.

In some embodiments, a template sequence or nucleic acid sample can be genomic DNA. In other embodiments, the template sequence or nucleic acid sample can be cDNA. In yet other embodiments, as in the case of simultaneous analysis of gene expression by RT-PCR, the template sequence or nucleic acid sample can be RNA. The DNA or RNA template sequence or nucleic acid sample can be extracted from any type of tissue including, for example, formalin-fixed paraffin-embedded tumor specimens.

In some embodiments, the target nucleic acid strand can be one present in a cell of a subject, such as a mammal (e.g., human), a plant, a fungus (e.g., a yeast), a protozoa, a bacterium, or a virus. For example, the target nucleic acid can be present in the genome of an organism of interest (e.g., on a chromosome) or on an extrachromosomal nucleic acid. In some embodiments, the target nucleic acid can be RNA, e.g., an mRNA. In some other embodiments, the target nucleic acid can be DNA (e.g., double-stranded DNA).

In particular embodiments, the target nucleic acid can be specific for the organism of interest, i.e., the target nucleic acid is not found in other organisms or not found in organisms similar to the organism of interest. In some embodiments, the target nucleic acid can be a viral nucleic acid. For example, the viral nucleic acid can be that in human immunodeficiency virus (HIV), an influenza virus (e.g., an influenza A virus, an influenza B virus, or an influenza C virus), or a dengue virus. The target nucleic acid can be present in a bacterium. In some embodiments, the target nucleic acid can be a protozoan nucleic acid. In some embodiments, the target nucleic acid is a fungal (e.g., yeast) nucleic acid.

In some preferred embodiments, the target nucleic acid can be a mammalian (e.g., human) nucleic acid. For example, the mammalian nucleic acid can be found in circulating tumor cells, epithelial cells, or fibroblasts. In other examples, the target nucleic acid is nucleic acids circulating freely in the blood of a subject, such as cell free nucleic acids (cfNA) or circulating tumor DNA (ctDNA) in the blood of a cancer patient. To best utilize the limited amount of cell free nucleic acids (cfNA) obtained from patient plasma, multiplex PCR chemistry and a universal PCR reaction program can be used. Multiplex assay allows simultaneous examination of an array of driver mutations, a requirement for a comprehensive lung cancer liquid biopsy.

Cell free DNA found in the blood and other bodily tissues can be used to detect and diagnose many genetic disorders. Numerous methods exist for non-invasive prenatal genetic diagnostics. Non-invasive prenatal genetic diagnoses can be performed on cell-free DNA, e.g., obtained from blood, from a patient. Cell-free DNA can also be used to detect or monitor the presence of tumor cells in patient.

To reach the goal of 5-day turnaround time recommended by CAP (Lindeman N I, Cagle P T, Beasley M B, et al. Molecular testing guideline for selection of lung cancer patients for EGFR and ALK tyrosine kinase inhibitors: guideline from the College of American Pathologists, International Association for the Study of Lung Cancer, and Association for Molecular Patho. Arch Pathol Lab Med. 2013; 137(6):828-860. doi:10.5858/arpa.2012-0720-OA), the library preparation procedure was optimized to under 4 hours. It is estimated that the turnaround time for the liquid biopsy is 4 days taking in account of sample logistics, sequencing, data analysis and interpretation.

In one example, the target strand is one containing a particular variant, such as single-nucleotide polymorphism (SNP) or a genetic mutation. Examples of such a mutation include a point mutation, a translocation, or an inversion.

In some embodiments, the compositions, methods, and/or kits disclosed herein can be used in detecting circulating cells in diagnosis. In one embodiment, the compositions, methods, and/or kits can be used to detect tumor cell DNA in blood for early cancer diagnosis. In some embodiments, the compositions, methods, and/or kits can be used for cancer or disease-associated genetic variation or somatic mutation detection and validation. In some embodiments, the compositions, methods, and/or kits can be used for genotyping tera-, tri- and di-allelic SNPs. In other embodiments, the compositions, methods, and/or kits can be used for identifying single or multiple nucleotide insertion or deletion mutations. In some embodiments, the compositions, methods, and/or kits can be used for DNA typing from mixed DNA samples for QC and human identification assays, cell line QC for cell contaminations, allelic gene expression analysis, virus typing/rare pathogen detection, mutation detection from pooled samples, detection of circulating tumor cells in blood, and/or prenatal diagnostics.

In some embodiments, when a target nucleic acid is an RNA, the sample can be subjected to a reverse transcriptase regimen to generate DNA template and the DNA template can then be sheared. In some embodiments, target RNA can be sheared before performing a reverse transcriptase regimen. In some embodiments, a sample comprising target RNA can be used in methods described herein using total nucleic acids extracted from either fresh or degraded specimens; without the need of genomic DNA removal for cDNA sequencing; without the need of ribosomal RNA depletion for cDNA sequencing; without the need of mechanical or enzymatic shearing in any of the steps; by subjecting the RNA for double-stranded cDNA synthesis using random hexamers.

In some embodiments, a known target nucleic acid can contain a fusion sequence resulting from a gene rearrangement. In some embodiments, methods described herein are suited for determining the presence and/or identity of a gene rearrangement. In some embodiments, identity of one portion of a gene rearrangement is previously known (e.g., the portion of a gene rearrangement that is to be targeted by the gene-specific primers) and the sequence of the other portion may be determined using methods disclosed herein. In some embodiments, a gene rearrangement can involve an oncogene. In some embodiments, a gene rearrangement can comprise a fusion oncogene.

In some embodiments, a target nucleic acid is present in or obtained from an appropriate sample (e.g., a food sample, environmental sample, biological sample e.g., blood sample, etc.). In some embodiments, the sample is a biological sample obtained from a subject. In some embodiments a sample can be a diagnostic sample obtained from a subject. In some embodiments, a sample can further comprise proteins, cells, fluids, biological fluids, preservatives, and/or other substances. By way of non-limiting example, a sample can be a cheek swab, blood, serum, plasma, sputum, cerebrospinal fluid, urine, tears, alveolar isolates, pleural fluid, pericardial fluid, cyst fluid, tumor tissue, tissue, a biopsy, saliva, an aspirate, or combinations thereof. In some embodiments, a sample can be obtained by resection or biopsy.

In some embodiments, the sample is freshly collected. In some embodiments, the sample is stored prior to being used in methods and compositions described herein. In some embodiments, the sample is an untreated sample. As used herein, “untreated sample” refers to a biological sample that has not had any prior sample pre-treatment except for dilution and/or suspension in a solution. In some embodiments, a sample is obtained from a subject and preserved or processed prior to being utilized in methods and compositions described herein. By way of non-limiting example, a sample can be embedded in paraffin wax, refrigerated, or frozen. A frozen sample can be thawed before determining the presence of a nucleic acid according to methods and compositions described herein. In some embodiments, the sample can be a processed or treated sample. Exemplary methods for treating or processing a sample include, but are not limited to, centrifugation, filtration, sonication, homogenization, heating, freezing and thawing, contacting with a preservative (e.g. anti-coagulant or nuclease inhibitor) and any combination thereof. In some embodiments, a sample can be treated with a chemical and/or biological reagent. Chemical and/or biological reagents can be employed to protect and/or maintain the stability of the sample or nucleic acid comprised by the sample during processing and/or storage. In addition, or alternatively, chemical and/or biological reagents can be employed to release nucleic acids from other components of the sample. By way of non-limiting example, a blood sample can be treated with an anti-coagulant prior to being utilized in methods and compositions described herein. Suitable methods and processes for processing, preservation, or treatment of samples for nucleic acid analysis may be used in the method disclosed herein. In some embodiments, a sample can be a clarified fluid sample, for example, by centrifugation. In some embodiments, a sample can be clarified by low-speed centrifugation (e.g. 3,000×g or less) and collection of the supernatant comprising the clarified fluid sample.

In some embodiments, a nucleic acid present in a sample can be isolated, enriched, or purified prior to being utilized in methods and compositions described herein. Suitable methods of isolating, enriching, or purifying nucleic acids from a sample may be used. For example, kits for isolation of genomic DNA from various sample types are commercially available (e.g. Catalog Nos. 51104, 51304, 56504, and 56404; Qiagen; Germantown, Md.). In some embodiments, methods described herein relate to methods of enriching for target nucleic acids, e.g., prior to sequencing of the target nucleic acids. In some embodiments, a sequence of one end of the target nucleic acid to be enriched is not known prior to sequencing. In some embodiments, methods described herein relate to methods of enriching specific nucleotide sequences prior to determining the nucleotide sequence using a next-generation sequencing technology. In some embodiments, methods of enriching specific nucleotide sequences do not comprise hybridization enrichment.

### Multiplex

Methods described herein can be employed in a multiplex format. In embodiments of methods described herein, multiplex applications can include determining the nucleotide sequence contiguous to one or more known target nucleotide sequences. As used herein, “multiplex amplification” refers to a process involving simultaneous amplification of more than one target nucleic acid in one reaction vessel. In some embodiments, methods involve subsequent determination of the sequence of the multiplex amplification products using one or more sets of primers. Multiplex can refer to the detection of between about 2-1,000 different target sequences in a single reaction. As used herein, multiplex refers to the detection of any range between 2 and 1000, e.g., 5-500, 25-1000, or 10-100 different target sequences in a single reaction, etc. The term “multiplex” as applied to PCR implies that there are primers specific for at least two different target sequences in the same PCR reaction.

In some embodiments, target nucleic acids in a sample, or separate portions of a sample, can be amplified with a plurality of primers (e.g., a plurality of first and second target-specific primers). In some embodiments, the plurality of primers (e.g., a plurality of first and second target-specific primers) can be present in a single reaction mixture, e.g. multiple amplification products can be produced in the same reaction mixture. In some embodiments, the plurality of primers (e.g., a plurality of sets of first and second target-specific primers) can specifically anneal to known target sequences comprised by separate genes. In some embodiments, at least two sets of primers (e.g., at least two sets of first and second target-specific primers) can specifically anneal to different portions of a known target sequence. In some embodiments, at least two sets of primers (e.g., at least two sets of first and second target-specific primers) can specifically anneal to different portions of a known target sequence comprised by a single gene. In some embodiments, at least two sets of primers (e.g., at least two sets of first and second target-specific primers) can specifically anneal to different exons of a gene comprising a known target sequence. In some embodiments, the plurality of primers (e.g., first target-specific primers) can comprise identical 5′ tag sequence portions.

In embodiments of methods described herein, multiplex applications can include determining the nucleotide sequence contiguous to one or more known target nucleotide sequences in multiple samples in one sequencing reaction or sequencing run. In some embodiments, multiple samples can be of different origins, e.g. from different tissues and/or different subjects. In such embodiments, primers can further comprise a barcode portion. In some embodiments, a primer with a unique barcode portion can be added to each sample and ligated to the nucleic acids therein; the samples can subsequently be pooled. In such embodiments, each resulting sequencing read of an amplification product will comprise a barcode that identifies the sample containing the template nucleic acid from which the amplification product is derived.

### Cancer-Related Uses

In some embodiments, the sample can be obtained from a subject in need of treatment for a disease associated with a genetic alteration, e.g. cancer or a hereditary disease. In some embodiments, a known target sequence is present in a disease-associated gene.

In some embodiments, a sample is obtained from a subject in need of treatment for cancer. In some embodiments, the sample comprises a population of tumor cells, e.g. at least one tumor cell. In some embodiments, the sample comprises a tumor biopsy, including but not limited to, untreated biopsy tissue or treated biopsy tissue (e.g. formalin-fixed and/or paraffin-embedded biopsy tissue).

In some embodiments of methods described herein, a determination of the sequence as disclosed herein can provide information relevant to treatment of disease. Thus, in some embodiments, methods disclosed herein can be used to aid in treating disease. In some embodiments, a sample can be from a subject in need of treatment for a disease associated with a genetic alteration. In some embodiments, the target sequence is a sequence of a disease-associated gene, e.g. an oncogene. In some embodiments, the target sequence can comprise a mutation or genetic abnormality which is disease-associated, e.g. a SNP, an insertion, a deletion, and/or a gene rearrangement. In some embodiments, a target sequence is comprised of a gene rearrangement product. In some embodiments, a gene rearrangement can be an oncogene, e.g. a fusion oncogene.

Certain treatments for cancer are particularly effective against tumors comprising certain oncogenes or mutations, e.g. a treatment agent which targets the action or expression of a given fusion oncogene can be effective against tumors comprising that fusion oncogene but not against tumors lacking the fusion oncogene. Methods described herein can facilitate a determination of specific sequences that reveal oncogene status (e.g. mutations, SNPs, and/or rearrangements). In some embodiments, methods described herein can further allow the determination of specific sequences when the sequence of a flanking region is known, e.g. methods described herein can determine the presence and identity of gene rearrangements involving known genes (e.g., oncogenes) in which the precise location and/or rearrangement partner are not known before methods described herein are performed.

In some embodiments, technology described herein relates to a method of treating cancer. Accordingly, in some embodiments, methods provided herein may involve detecting, in a tumor sample obtained from a subject in need of treatment for cancer, the presence of one or more oncogene rearrangements; and administering a cancer treatment which is effective against tumors having any of the detected oncogene rearrangements. In some embodiments, technology described herein relates to a method of determining if a subject in need of treatment for cancer will be responsive to a given treatment. Accordingly, in some embodiments, methods provided herein may involve detecting, in a tumor sample obtained from a subject, the presence of an oncogene rearrangement, in which the subject is determined to be responsive to a treatment targeting an oncogene rearrangement product if the presence of the oncogene rearrangement is detected.

The system and method disclosed in this invention are particularly useful in the areas of (a) early cancer detection from tissue biopsies and bodily fluids such as plasma or serum; (b) assessment of residual disease after surgery or radio/chemotherapy; (c) disease staging and molecular profiling for prognosis or tailoring therapy to individual patients; and (d) monitoring of therapy outcome and cancer remission/relapse.

Cancer can include, but is not limited to, carcinoma, including adenocarcinoma, lymphoma, blastoma, melanoma, sarcoma, leukemia, squamous cell cancer, small-cell lung cancer, non-small cell lung cancer, gastrointestinal cancer, Hodgkin's and non-Hodgkin's lymphoma, pancreatic cancer, glioblastoma, basal cell carcinoma, biliary tract cancer, bladder cancer, brain cancer including glioblastomas and medulloblastomas; breast cancer, cervical cancer, choriocarcinoma; colon cancer, colorectal cancer, endometrial carcinoma, endometrial cancer; esophageal cancer, gastric cancer; various types of head and neck cancers, intraepithelial neoplasms including Bowen's disease and Paget's disease; hematological neoplasms including acute lymphocytic and myelogenous leukemia; Kaposi's sarcoma, hairy cell leukemia; chromic myelogenous leukemia, AIDS-associated leukemias and adult T-cell leukemia lymphoma; kidney cancer such as renal cell carcinoma, T-cell acute lymphoblastic leukemia/lymphoma, lymphomas including Hodgkin's disease and lymphocytic lymphomas; liver cancer such as hepatic carcinoma and hepatoma, Merkel cell carcinoma, melanoma, multiple myeloma; neuroblastomas; oral cancer including squamous cell carcinoma; ovarian cancer including those arising from epithelial cells, sarcomas including leiomyosarcoma, rhabdomyosarcoma, liposarcoma, fibROS 1 arcoma, and osteosarcoma; pancreatic cancer; skin cancer including melanoma, stromal cells, germ cells and mesenchymal cells; pROS ltate cancer, rectal cancer; vulval cancer, renal cancer including adenocarcinoma; testicular cancer including germinal tumors such as seminoma, non-seminoma (teratomas, choriocarcinomas), stromal tumors, and germ cell tumors; thyroid cancer including thyroid adenocarcinoma and medullar carcinoma; esophageal cancer, salivary gland carcinoma, and Wilms' tumors. In some embodiments, the cancer can be lung cancer, such as NSCLC.

NSCLC

Cancer deaths in the United States are projected at 589,430 in 2015 (Siegel R L, Miller K D, Jemal A. Cancer statistics, 2015. CA Cancer J Clin. 2015; 65(1):5-29). Among the cancers, non-small cell lung cancer (NSCLC), the number one cause of cancer mortality, accounts for 22.8%. Platinum-based combination chemotherapy moderately improves advanced NSCLC patient survival by 9% at 12 months compared to supportive care alone (Spiro S G, Rudd R M, Souhami R L, et al. Chemotherapy versus supportive care in advanced non-small cell lung cancer: improved survival without detriment to quality of life. Thorax. 2004; 59(10):828-836). In comparison, targeted therapy incorporates tumor genotyping into therapeutic decision-making and has greatly improved upon treatment efficacy. For instance, gefitinib, a small-molecule tyrosine kinase inhibitor (TKI) that targets epidermal growth factor receptor (EGFR), has a response rate up to 75% compared to 20% by typical chemotherapy and median survival of 28.2 months versus 10.3 months. See, e.g., Paez J G, Ja P A, Tracy S, et al. EGFR Mutations in Lung Cancer: Correlation with Clinical Response to Gefitinib Therapy. 2004; 304(June):1497-1501, and Barr Kumarakulasinghe N, Zanwijk N Van, Soo R a. Molecular targeted therapy in the treatment of advanced stage non-small cell lung cancer (NSCLC). Respirology. February 2015. However, targeted therapy does not benefit all NSCLC patients. The high efficacy relies on the existence of actionable oncogenic driver mutations in patients. These mutations are molecular abnormalities that initiate or maintain the neoplastic process and can be negated by agents directed against each genomic alteration. Without targetable neoplastic process, gefitinib has a miniscule 0-6.6% response rate in NSCLC patients with a wild type EGFR gene, but an exceptional 75% response rate in patients with EGFR mutations. See, e.g., Douillard J-Y, Shepherd F a, Hirsh V, et al. Molecular predictors of outcome with gefitinib and docetaxel in previously treated non-small-cell lung cancer: data from the randomized phase III INTEREST trial. J Clin Oncol. 2010; 28(5):744-752; Hirsch F R, Varella-Garcia M, Bunn P a., et al. Molecular Predictors of Outcome With Gefitinib in a Phase III Placebo-Controlled Study in Advanced Non-Small-Cell Lung Cancer. J Clin Oncol. 2006; 24(31):5034-5042; Maruyama R, Nishiwaki Y, Tamura T, et al. Phase III study, V-15-32, of gefitinib versus docetaxel in previously treated Japanese patients with non-small-cell lung cancer. J Clin Oncol. 2008; 26(26):4244-4252; and Mok T, Wu Y. Gefitinib or carboplatin-paclitaxel in pulmonary adenocarcinoma. Engl J 2009:947-957. Driver mutation tests are therefore required prior to targeted therapy.

Tissue biopsy has been the primary source for mutation identification. However, it is not ideal for NSCLC patients. Approximately 75% of the NSCLC cases are advanced at diagnosis (see, e.g., Reade C, Ganti A. EGFR targeted therapy in non-small cell lung cancer: potential role of cetuximab. Biol targets Ther. 2009:215-224) but solid biopsy has an inherent disadvantage in these cases of detecting intertumoral and intratumoral heterogeneity, which often leads to drug resistance. In fact, the presence of tumor heterogeneity is a major challenge in developing effective cancer treatment using targeted therapies (Yancovitz M, Litterman A, Yoon J, et al. Intra- and inter-tumor heterogeneity of BRAF(V600E) mutations in primary and metastatic melanoma. PLoS One. 2012; 7(1):e29336). Liquid biopsy as an attractive approach has the potential to capture a comprehensive profile of genomic alterations and thus allows delivery of effective targeted therapy. Moreover, a liquid biopsy is easily repeatable, which makes it possible to monitor the tumor dynamics and, thus, to guide drug changes during therapy. Liquid biopsy can also potentially be used after surgery or therapy to measure minimal residual disease that may result in recurrence. See, e.g., Diehl F, Schmidt K, Choti M a, et al. Circulating mutant DNA to assess tumor dynamics. Nat Med. 2008; 14(9):985-990. In addition, it can be an important companion tool in the follow-up care to detect early signs of relapse. See, e.g., Misale S, Yaeger R, Hobor S, et al. Emergence of KRAS mutations and acquired resistance to anti-EGFR therapy in colorectal cancer. Nature. 2012; 486(7404):532-536 and Diaz L a, Williams R T, Wu J, et al. The molecular evolution of acquired resistance to targeted EGFR blockade in colorectal cancers. Nature. 2012; 486(7404):537-540. However, due to the low presence of circulating tumor nucleic acids (ctNA) and circulating tumor cells, aggravated by artificial errors introduced by detection methods, liquid biopsy has not been used by physicians in daily practice.

The system and method disclosed in this invention can be used as a reliable and rapid liquid biopsy assay for late stage NSCLC patients. The barriers to develop such a reliable and rapid assay are twofold and no currently available platform yet overcomes both.

The first one is sensitivity. The presence of circulating tumor DNA (ctDNA) even in late stage cancer patients can be extremely low. Newman et al observed a range of 0.04% to 3.2% ctDNA in plasma of advanced stage NSCLC patients. See Newman A M, Bratman S V, To J, et al. An ultrasensitive method for quantitating circulating tumor DNA with broad patient coverage. Nat Med. 2014; 20(5):548-554. Quantitative PCR (qPCR), a commonly used technology, can reach limit of detection (LOD) of about 1% and Next Generation Sequencing (NGS) can mostly reach a 1-2% LOD. Digital PCR in this aspect shows the most promising advance, with a low LOD of 0.01%. See, e.g., Detection of rare mutations in blood samples by droplet digital PC at www.bio-rad.com/webroot/web/pdf/lsr/literature/Bulletin_6317.pdf.

The second is multiplex capability. The number of discovered actionable mutations and the limited volume of blood sample render a compilation of singleplex assays unsuitable for liquid biopsy. Therefore, a crucial feature of a functional liquid biopsy is the ability to examine multiple driver mutations in parallel from a single plasma DNA sample. NGS is the only mature platform currently offering sufficient multiplex capability.

The liquid biopsy disclosed herein focuses on the identification of actionable oncogenic mutations to guide therapy selection. Approximately 75% of the NSCLC are metastatic or advanced at diagnosis (see, e.g., Reade C, Ganti A. EGFR targeted therapy in non-small cell lung cancer: potential role of cetuximab. Biol Targets Ther. 2009:215-224) and are eligible for targeted therapy. In these patients tumor heterogeneity is present at a high level. A primary objective here is to test for actionable mutations in a heterogenic tumor cell population and then closely monitor tumor response to therapy and the rise of molecular resistance. Patients under follow-up care post-treatment also greatly benefit from liquid biopsy. Studies have found that molecular tests can detect relapses months before radiologic examination. See, e.g., Misale S, Yaeger R, Hobor S, et al. Emergence of KRAS mutations and acquired resistance to anti-EGFR therapy in colorectal cancer. Nature. 2012; 486(7404):532-536, and Diaz L a, Williams R T, Wu J, et al. The molecular evolution of acquired resistance to targeted EGFR blockade in colorectal cancers. Nature. 2012; 486(7404):537-540.

The system and method disclosed herein can be used to enrich and detect a number of mutations at certain hotspots including those encoding EGFR T790M, EGFR L858R, BRAF V600E, BRAF V600K, BRAF V600D, BRAF V600G, BRAF V600A, BRAF V600R, and KRASG12V.

EGFR T790M mutation is a frequently acquired mutation in patients on TKI targeted therapy that results in an amino acid substitution from threonine to methionine at EGFR position 790. This mutated residue increases affinity to ATP and outcompetes the binding of the inhibitors. See, e.g., Yun C-H, Mengwasser K E, Toms A V, et al. The T790M mutation in EGFR kinase causes drug resistance by increasing the affinity for ATP. Proc Natl Acad Sci USA. 2008; 105(6):2070-2075. Patients start to show reduced sensitivity to TKI with as low as 5% of cancer cells that acquired this mutation. See, e.g., Lindeman N I, Cagle P T, Beasley M B, et al. Molecular testing guideline for selection of lung cancer patients for EGFR and ALK tyrosine kinase inhibitors: guideline from the College of American Pathologists, International Association for the Study of Lung Cancer, and Association for Molecular Patho. Arch Pathol Lab Med. 2013; 137(6):828-860. Therefore, it is necessary to closely monitor the emergence of T790M. The detection of T790M may assist a physician's decision making of switching drug to the third-generation EGFR TKIs or a hiatus during therapy. See, e.g., Watanabe S, Tanaka J, Ota T, et al. Clinical responses to EGFR-tyrosine kinase inhibitor retreatment in non-small cell lung cancer patients who benefited from prior effective gefitinib therapy: a retrospective analysis. BMC Cancer. 2011; 11(1):1. EGFR L858R is an oncogenic driver that accounts for 43% of all EGFR activated lung cancer. See, e.g., Mitsudomi T, Yatabe Y. Epidermal growth factor receptor in relation to tumor development: EGFR gene and cancer. FEBS J. 2010; 277(2):301-308. doi:10.1111/j.1742-4658.2009.07448.x.

In addition to the above point mutations, other nucleic acid variants or mutations can be enriched and/or amplified by the method and system of this invention. Examples of the nucleic acid variants include those described in PCT/US2016/057805 and U.S. Application No. 62/244,279, such as those in the tables below. The contents of PCT/US2016/057805 and U.S. Application No. 62/244,279 are incorporated by reference in its entirety.

TABLE A
List of actionable mutations to be enriched

| | | | |
|---|---|---|---|
| | EGFR | EGFR | EGFR |
| EGFR c.2155 | c.2238_2252del15 | c.2240_2257del18 | c.2369 |
| EGFR c.2156 | EGFR c.2239_2247 | EGFR c.2307_2308 | EGFR |
| | delTTAAGAGAA | insGCCAGCGTG | c.2573 |
| | (SEQ ID NO: 3) | (SEQ ID NO: 4) | |
| EGFR | EGFR c.2239_2248 | EGFR c.2308_2309 | EGFR |
| c.2235_2246del12 | TTAAGAGAAG > C | insCCAGCGTGG | c.2582 |
| | (SEQ ID NO: 5) | (SEQ ID NO: 6) | |
| EGFR | EGFR | EGFR | BRAF |
| c.2235_2249del15 | c.2239_2251 > C | c.2231_2232ins18 | c.1799 |
| EGFR | EGFR | EGFR | KRAS c.34 |
| c.2236_2250del15 | c.2239_2253del15 | c.2234_2235ins18 | |
| EGFR | EGFR | EGFR | KRAS c.35 |
| c.2237_2251del15 | c.2239_2256del18 | c.2236_2237ins18 | |
| EGFR | EGFR | EGFR | KRAS c.37 |
| c.2237_2253 > TTGCT | c.2239_2257 > GT | c.2232_2233ins18 | |
| EGFR | EGFR | EGFR c.2303 | KRAS c.38 |
| c.2237_2255 > T | c.2240_2254del15 | | |

TABLE B
List of ALK and ROS1 fusion junctions

| | | |
|---|---|---|
| EML4_E13- | EML4_E14; del12- | SLC34A2_E13- |
| ALK-E20 | ALK-E20 | ROS1_E32 |
| EML4_E20- | KIF5B_E24- | GOPC_E8- |
| ALK-E20 | ALK_E20 | ROS1_E35 |
| EML4_E6- | KIF5B_E15- | GOPC_E4- |
| ALK-E20 | ALK_E20 | ROS1_E36 |
| EML4_E6; | KIF5B_E17- | EZR_E10- |
| ins33-ALK-E20 | ALK_E20 | ROS1_E34 |
| EML4_E14; | TFG_E4- | SDC4_E2- |
| ins11del49- | ALK_E20 | ROS1_E32 |
| ALK-E20 | | |
| EML4_E2- | CD74_E6- | SDC4_E4- |
| ALK-E20 | ROS1_E34 | ROS1_E32 |
| EML4_E2; | CD74_E6- | TPM3_E8- |
| ins117-ALK-E20 | ROS1_E32 | ROS1_E35 |
| EML4_E13; | SLC34A2_E4- | LRIG3_E16- |
| ins69-ALK-E20 | ROS1_E32 | ROS1_E35 |

The system and method disclosed herein provide best-in-class liquid biopsy products. Compared to in silico enhanced liquid biopsies that may take up the full capacity of an Illumina HiSeq by a single biopsy sample (Sullivan M. Guardant Health takes another $50M for “liquid biopsy” cancer test. 2015. http://venturebeat.com/2015/02/03/guardant-health-takes-another-50m-for-ground-breaking-liquid-biopsy-test/), the assay disclosed herein reduces the presence of non-mutated DNA in vitro to allow more sensitive detection of the oncogenic mutations and is able to use the same HiSeq sequencing capacity to process 10,000 samples.

In some embodiments, methods described herein relate to treating a subject having or diagnosed as having, e.g. cancer with a treatment for cancer. Subjects having cancer can be identified by a physician using current methods of diagnosing cancer. For example, symptoms and/or complications of lung cancer which characterize these conditions and aid in diagnosis are well known in the art and include but are not limited to, weak breathing, swollen lymph nodes above the collarbone, abnormal sounds in the lungs, dullness when the chest is tapped, and chest pain. Tests that may aid in a diagnosis of, e.g. lung cancer include, but are not limited to, x-rays, blood tests for high levels of certain substances (e.g. calcium), CT scans, and tumor biopsy. A family history of lung cancer, or exposure to risk factors for lung cancer (e.g. smoking or exposure to smoke and/or air pollution) can also aid in determining if a subject is likely to have lung cancer or in making a diagnosis of lung cancer.

In addition to lung cancer, the invention disclosed herein can also be used to detect markers for other malignancy. Further non-limiting examples of applications of invention described herein include detection of hematological malignancy markers and panels thereof (e.g. including those to detect genomic rearrangements in lymphomas and leukemias), detection of sarcoma-related genomic rearrangements and panels thereof; and detection of IGH/TCR gene rearrangements and panels thereof for lymphoma testing.

### Compositions and Kits

The invention encompasses a composition or reaction mixture comprising the aforementioned adapters, primers and blockers. The composition can further comprise one or more reagents selected from the group consisting of a nucleic acid polymerase, extension nucleotides, and a detecting agent.

The detecting agent can be a nucleotide probe, such as a molecular beacon probe or a Yin-Yang probe that is labeled with a fluorophore and a quencher. See e.g., U.S. Pat. Nos. 5,925,517, 6,103,476, 6,150,097, 6,270,967, 6,326,145, and 7,799,522. The composition can also comprise, in addition to the above reagents, one or more of: a salt, e.g., NaCl, MgCl₂, KCl, MgSO₄; a buffering agent, e.g., a Tris buffer, N-(2-Hydroxyethyl)piperazine-N′-(2-ethanesulfonic acid) (HEPES), 2-(N-Morpholino)ethanesulfonic acid (MES), MES sodium salt, 3-(N-Morpholino)propanesulfonic acid (MOPS), N-tris[Hydroxymethyl]methyl-3-aminopro-panesulfonic acid (TAPS); a solubilizing agent; a detergent, e.g., a non-ionic detergent such as Tween-20; a nuclease inhibitor; and the like.

The invention encompasses kits and diagnostic systems for conducting amplification, enrichment, and/or for detection of a target sequence. To that end, one or more of the reaction components for the methods disclosed herein can be supplied in the form of a kit for use in the enrichment and detection of a target nucleic acid strand. In such a kit, an appropriate amount of one or more reaction components is provided in one or more containers or held on a substrate (e.g., by electrostatic interactions or covalent bonding).

A kit containing reagents for performing amplification or enrichment or sequencing (such as those for NGS or Sanger sequencing) of a target nucleic acid sequence using the methods described herein may include one or more of the followings: one or more adapters, a forward primer, a reverse primer, one or more blockers, a nucleic acid polymerase, extension nucleotides, and detection probes. Examples of additional components of the kits include, but are not limited to, one or more different polymerases, one or more primers that are specific for a control nucleic acid or for a target nucleic acid, one or more probes that are specific for a control nucleic acid or for a target nucleic acid, buffers for polymerization reactions (in 1× or concentrated forms), and one or more dyes or fluorescent molecules for detecting polymerization products. The kit may also include one or more of the following components: supports, terminating, modifying or digestion reagents, osmolytes, and an apparatus for detecting a detection probe.

The reaction components used in an amplification and/or detection process may be provided in a variety of forms. For example, the components (e.g., enzymes, nucleotide triphosphates, adaptors, blockers, and/or primers) can be suspended in an aqueous solution or as a freeze-dried or lyophilized powder, pellet, or bead. In the latter case, the components, when reconstituted, form a complete mixture of components for use in an assay.

A kit or system may contain, in an amount sufficient for at least one assay, any combination of the components described herein, and may further include instructions recorded in a tangible form for use of the components. In some applications, one or more reaction components may be provided in pre-measured single use amounts in individual, typically disposable, tubes or equivalent containers. With such an arrangement, the sample to be tested for the presence of a target nucleic acid can be added to the individual tubes and amplification carried out directly. The amount of a component supplied in the kit can be any appropriate amount, and may depend on the target market to which the product is directed. General guidelines for determining appropriate amounts may be found in, for example, Joseph Sambrook and David W. Russell, Molecular Cloning: A Laboratory Manual, 3rd edition, Cold Spring Harbor Laboratory Press, 2001; and Frederick M. Ausubel, Current Protocols in Molecular Biology, John Wiley & Sons, 2003.

The kits of the invention can comprise any number of additional reagents or substances that are useful for practicing a method of the invention. Such substances include, but are not limited to: reagents (including buffers) for lysis of cells, divalent cation chelating agents or other agents that inhibit unwanted nucleases, control DNA for use in ensuring that the enzyme complexes and other components of reactions are functioning properly, DNA fragmenting reagents (including buffers), amplification reaction reagents (including buffers), and wash solutions. The kits of the invention can be provided at any temperature. For example, for storage of kits containing protein components or complexes thereof in a liquid, it is preferred that they are provided and maintained below 0° C., preferably at or below −20° C., or otherwise in a frozen state.

The container(s) in which the components are supplied can be any conventional container that is capable of holding the supplied form, for instance, microfuge tubes, ampoules, bottles, or integral testing devices, such as fluidic devices, cartridges, lateral flow, or other similar devices. The kits can include either labeled or unlabeled nucleic acid probes for use in detection of target nucleic acids. In some embodiments, the kits can further include instructions to use the components in any of the methods described herein, e.g., a method using a crude matrix without nucleic acid extraction and/or purification. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like).

A system, in addition to containing kit components, may further include instrumentation for conducting an assay, e.g. a luminometer for detecting a signal from a labeled probe.

Instructions, such as written directions or videotaped demonstrations detailing the use of the kits or system of the present invention, are optionally provided with the kit or systems. In a further aspect, the present invention provides for the use of any composition or kit herein, for the practice of any method or assay herein, and/or for the use of any apparatus or kit to practice any assay or method herein.

Optionally, the kits or systems of the invention further include software to expedite the generation, analysis and/or storage of data, and to facilitate access to databases. The software includes logical instructions, instructions sets, or suitable computer programs that can be used in the collection, storage and/or analysis of the data. Comparative and relational analysis of the data is possible using the software provided.

All of the above-described methods, reagents, and systems provide a variety of diagnostic tools which permit a blood-based, non-invasive assessment of disease status in a subject. Use of these methods, reagents, and systems in diagnostic tests, which may be coupled with other screening tests, such as a chest X-ray or CT scan, increase diagnostic accuracy and/or direct additional testing. In other aspects, the inventions described herein permit the prognosis of disease, monitoring response to specific therapies, and regular assessment of the risk of recurrence. The inventions described herein also permit the evaluation of changes in diagnostic signatures present in pre-surgery and post therapy samples and identifies a gene expression profile or signature that reflects tumor presence and may be used to assess the probability of recurrence.

A significant advantage of the methods of this invention over existing methods is that they are able to characterize the disease state from a minimally-invasive procedure, e.g., by taking a blood sample without isolating cancer cells. In contrast current practice for classification of cancer tumors from gene expression profiles depends on a tissue sample, usually a sample from a tumor. In the case of very small tumors, a biopsy is problematic and clearly if no tumor is known or visible, a sample from it is impossible. No purification or isolation of tumor is required, as is the case when tumor samples are analyzed. Blood samples have an additional advantage, which is that the material is easily prepared and stabilized for later analysis, which is important when messenger RNA is to be analyzed.

### Definitions

A “nucleic acid” refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA.

As used herein, the term “target nucleic acid” or “target” refers to a nucleic acid containing a target nucleic acid sequence. A target nucleic acid may be single-stranded or double-stranded, and often is DNA, RNA, a derivative of DNA or RNA, or a combination thereof. A “target nucleic acid sequence,” “target sequence” or “target region” means a specific sequence comprising all or part of the sequence of a single-stranded nucleic acid. A target sequence may be within a nucleic acid template, which may be any form of single-stranded or double-stranded nucleic acid. A template may be a purified or isolated nucleic acid, or may be non-purified or non-isolated.

The term “allele” refers generally to alternative DNA sequences at the same physical locus on a segment of DNA, such as, for example, on homologous chromosomes. An allele can refer to DNA sequences which differ between the same physical locus found on homologous chromosomes within a single cell or organism or which differ at the same physical locus in multiple cells or organisms (“allelelic variant”). In some instances, an allele can correspond to a single nucleotide difference at a particular physical locus. In other embodiments an allele can correspond to nucleotide (single or multiple) insertion or deletion.

As used herein, the term “rare allelic variant” refers to a target polynucleotide present at a lower level in a sample as compared to an alternative allelic variant. The rare allelic variant may also be referred to as a “minor allelic variant” and/or a “mutant allelic variant.” For instance, the rare allelic variant may be found at a frequency less than 1/10, 1/100, 1/1,000, 1/10,000, 1/100,000, 1/1,000,000, 1/10,000,000, 1/100,000,000 or 1/1,000,000,000 compared to another allelic variant for a given SNP or gene. Alternatively, the rare allelic variant can be, for example, less than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, or 1,000,000 copies per 1, 10, 100, 1,000 micro liters of a sample or a reaction volume.

As used herein, the terms “abundant allelic variant” may refer to a target polynucleotide present at a higher level in a sample as compared to an alternative allelic variant. The abundant allelic variant may also be referred to as a “major allelic variant” and/or a “wild type allelic variant.” For instance, the abundant allelic variant may be found at a frequency greater than 10×, 100×, 1,000×, 10,000×, 100,000×, 1,000,000×, 10,000,000×, 100,000,000×. or 1,000,000,000× compared to another allelic variant for a given SNP or gene. Alternatively, the abundant allelic variant can be, for example, greater than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, 1,000,000 copies per 1, 10, 100, 1,000 micro liters of a sample or a reaction volume.

As used herein the term “amplification” and its variants includes any process for producing multiple copies or complements of at least some portion of a polynucleotide, said polynucleotide typically being referred to as a “template.” The template polynucleotide can be single stranded or double stranded. Amplification of a given template can result in the generation of a population of polynucleotide amplification products, collectively referred to as an “amplicon.” The polynucleotides of the amplicon can be single stranded or double stranded, or a mixture of both. Typically, the template will include a target sequence, and the resulting amplicon will include polynucleotides having a sequence that is either substantially identical or substantially complementary to the target sequence. In some embodiments, the polynucleotides of a particular amplicon are substantially identical, or substantially complementary, to each other; alternatively, in some embodiments the polynucleotides within a given amplicon can have nucleotide sequences that vary from each other. Amplification can proceed in linear or exponential fashion, and can involve repeated and consecutive replications of a given template to form two or more amplification products. Some typical amplification reactions involve successive and repeated cycles of template-based nucleic acid synthesis, resulting in the formation of a plurality of daughter polynucleotides containing at least some portion of the nucleotide sequence of the template and sharing at least some degree of nucleotide sequence identity (or complementarity) with the template. In some embodiments, each instance of nucleic acid synthesis, which can be referred to as a “cycle” of amplification, includes creating free 3′ end (e.g., by nicking one strand of a dsDNA) thereby generating a primer and primer extension steps; optionally, an additional denaturation step can also be included wherein the template is partially or completely denatured. In some embodiments, one round of amplification includes a given number of repetitions of a single cycle of amplification. For example, a round of amplification can include 5, 10, 15, 20, 25, 30, 35, 40, 50, 75, 100 or more repetitions of a particular cycle. In one exemplary embodiment, amplification includes any reaction wherein a particular polynucleotide template is subjected to two consecutive cycles of nucleic acid synthesis. The synthesis can include template-dependent nucleic acid synthesis.

The term “blocker” (also referred to herein as “oligonucleotide blocker,” “blocking oligo,” “blocker probe,” or “oligo blocker”) refers to a strand of nucleic acid or an oligonucleotide capable of hybridizing to a strand of DNA comprising a particular allelic variant which is located on the same, opposite or complementary strand as that bound by a primer (either a forward primer or a reverse primer), and reduces or prevents amplification of that particular allelic variant. A blocker can be designed, for example, so as to tightly bind to a wild type allele (e.g., abundant allelic variant) in order to suppress amplification of the wild type allele while amplification is allowed to occur on the same or opposing strand comprising a mutant allele (e.g., rare allelic variant) by extension of a primer.

The term “primer” or “primer oligonucleotide” refers to a strand of nucleic acid or an oligonucleotide capable of hybridizing to a template nucleic acid and acting as the initiation point for incorporating extension nucleotides according to the composition of the template nucleic acid for nucleic acid synthesis. A “target-specific primer” or “gene-specific primer” refers to a strand of nucleic acid or an oligonucleotide capable of hybridizing to a portion of a target nucleic acid or gene of interest. An “adapter primer” refers to a primer that is specific for an adapter as disclosed herein, but not the target nucleic acid or gene of interest.

As used herein, “specific” when used in the context of a primer specific for a target nucleic acid refers to a level of complementarity between the primer and the target such that there exists an annealing temperature at which the primer will anneal to and mediate amplification of the target nucleic acid and will not anneal to or mediate amplification of non-target sequences present in a sample.

As used herein, “amplified product,” “amplification product,” “amplified molecule,” or “amplicon” refers to oligonucleotides resulting from an amplification reaction that are copies of a portion of a particular target nucleic acid template strand and/or its complementary sequence, which correspond in nucleotide sequence to the template nucleic acid sequence and/or its complementary sequence. An amplification product can further comprise sequence specific to the primers and which flanks sequence which is a portion of the target nucleic acid and/or its complement. An amplified product, as described herein will generally be double-stranded DNA, although reference can be made to individual strands thereof.

In some embodiments, primers may contain additional sequences such as an identifier sequence (e.g., a barcode, an index), sequencing primer hybridization sequences (e.g., Rd1), and adapter sequences. In some embodiments the adapter sequences are sequences used with a next generation sequencing system. In some embodiments, the adapter sequences are P5 and P7 sequences for Illumina-based sequencing technology. In some embodiments, the adapter sequences are P1 and A compatible with Ion Torrent sequencing technology.

As used herein, a “barcode,” “molecular barcode,” “molecular barcode tag” and “index” may be used interchangeably, generally referring to a nucleotide sequence of a nucleic acid that is useful as an identifier, such as, for example, a source identifier, location identifier, date or time identifier (e.g., date or time of sampling or processing), or other identifier of the nucleic acid. In some embodiments, such barcode or index sequences are useful for identifying different aspects of a nucleic acid that is present in a population of nucleic acids. In some embodiments, barcode or index sequences may provide a source or location identifier for a target nucleic acid. For example, a barcode or index sequence may serve to identify a patient from whom a nucleic acid is obtained. In some embodiments, barcode or index sequences enable sequencing of multiple different samples on a single reaction (e.g., performed in a single flow cell). In some embodiments, an index sequence can be used to orientate a sequence imager for purposes of detecting individual sequencing reactions. In some embodiments, a barcode or index sequence may be 2 to 25 nucleotides in length, 2 to 15 nucleotides in length, 2 to 10 nucleotides in length, 2 to 6 nucleotides in length. In some embodiments, a barcode or index comprise at least 2, 3, 4, 5, 6, 7, 8, 9, 10, 11, 12, 13, 14, 15, 16, 17, 18, 19, 20, 21, 22, 23, 24, or at least 25 nucleotides.

“Extension nucleotides” refer to any nucleotide capable of being incorporated into an extension product during amplification, i.e., DNA, RNA, or a derivative if DNA or RNA, which may include a label.

As used herein, the term “modified base” refers generally to any modification of a base or the chemical linkage of a base in a nucleic acid that differs in structure from that found in a naturally occurring nucleic acid. Such modifications can include changes in the chemical structures of bases or in the chemical linkage of a base in a nucleic acid, or in the backbone structure of the nucleic acid. (See, e.g., Latorra, D. et al., Hum Mut 2003, 2:79-85. Nakiandwe, J. et al., Plant Method 2007, 3:2.).

The term “detection probe” refers to an oligonucleotide having a sequence sufficiently complementary to its target sequence to form a probe:target hybrid stable for detection under stringent hybridization conditions. A probe is typically a synthetic oligomer that may include bases complementary to sequence outside of the targeted region which do not prevent hybridization under stringent hybridization conditions to the target nucleic acid. A sequence non-complementary to the target may be a homopolymer tract (e.g., poly-A or poly-T), promoter sequence, restriction endonuclease recognition sequence, or sequence to confer desired secondary or tertiary structure (e.g., a catalytic site or hairpin structure), or a tag region which may facilitate detection and/or amplification. “Stable” or “stable for detection” means that the temperature of a reaction mixture is at least 2° C. below the melting temperature (Tm) of a nucleic acid duplex contained in the mixture, more preferably at least 5° C. below the Tm, and even more preferably at least 10° C. below the Tm.

Hybridization” or “hybridize” or “anneal” refers to the ability of completely or partially complementary nucleic acid strands to come together under specified hybridization conditions in a parallel or preferably antiparallel orientation to form a stable double-stranded structure or region (sometimes called a “hybrid”) in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

“Preferentially hybridize” means that under stringent hybridization conditions, nucleic acids or oligonucleotides (e.g., primers, blockers, or probes) can hybridize to their target nucleic acid sequence to form stable hybrids, e.g., to indicate the presence of at least one sequence or organism of interest in a sample. A nucleic acid hybridizes to its target nucleic acid specifically, i.e., to a sufficiently greater extent than to a non-target nucleic acid to accurately detect the presence (or absence) of the intended target sequence. Preferential hybridization generally refers to at least a 10-fold difference between target and non-target hybridization signals in a sample.

The term “stringent hybridization conditions” or “stringent conditions” means conditions in which a nucleic acid or oligomer hybridizes specifically to its intended target nucleic acid sequence and not to another sequence. Stringent conditions may vary depending on well-known factors, e.g., GC content and sequence length, and may be predicted or determined empirically using standard methods well known to one of ordinary skill in molecular biology (e.g., Sambrook, J. et al., 1989, Molecular Cloning, A Laboratory Manual, 2nd ed., Ch. 11, pp. 11.47-11.57, (Cold Spring Harbor Laboratory Press, Cold Spring Harbor, N.Y.)).

“Substantially homologous” or “substantially corresponding” means a probe, nucleic acid, or oligonucleotide has a sequence of at least 10, 20, 30, 40, 50, 100, 150, 200, 300, 400, or 500 contiguous bases that is at least 80% (preferably at least 85%, 90%, 95%, 96%, 97%, 98%, and 99%, and most preferably 100%) identical to contiguous bases of the same length in a reference sequence. Homology between sequences may be expressed as the number of base mismatches in each set of at least 10 contiguous bases being compared.

As used herein, the term “complementary” refers to the ability of nucleotides to form hydrogen-bonded base pairs. In some embodiment, complementary refers to hydrogen-bonded base pair formation preferences between the nucleotide bases G, A, T, C and U, such that when two given polynucleotides or polynucleotide sequences anneal to each other, A pairs with T and G pairs with C in DNA, and G pairs with C and A pairs with U in RNA. “Substantially complementary” means that an oligonucleotide has a sequence containing at least 10, 20, 30, 40, 50, 100, 150, 200, 300, 400, or 500 contiguous bases that are at least 80% (preferably at least 85%, 90%, 95%, 96%, 97%, 98%, and 99%, and most preferably 100%) complementary to contiguous bases of the same length in a target nucleic acid sequence. Complementarity between sequences may be expressed a number of base mismatches in each set of at least 10 contiguous bases being compared. As used herein, “substantially identical” refers to a nucleic acid molecule or portion thereof having at least 90% identity over the entire length of the molecule or portion thereof with a second nucleotide sequence, e.g. 90% identity, 95% identity, 98% identity, 99% identity, or 100% identity.

As used herein, the term “subject” refers to any organism having a genome, preferably, a living animal, e.g., a mammal, which has been the object of diagnosis, treatment, observation or experiment. Examples of a subject can be a human, a livestock animal (beef and dairy cattle, sheep, poultry, swine, etc.), or a companion animal (dogs, cats, horses, etc).

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. lung cancer. The term “treating” includes reducing or alleviating at least one adverse effect or symptom of a condition, disease or disorder associated with a condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The term “biological sample” refers to a sample obtained from an organism (e.g., patient) or from components (e.g., cells) of an organism. The sample may be of any biological tissue, cell(s) or fluid. The sample may be a “clinical sample” which is a sample derived from a subject, such as a human patient or veterinary subject. Such samples include, but are not limited to, saliva, sputum, blood, blood cells (e.g., white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample.” A biological sample may also include a substantially purified or isolated protein, membrane preparation, or cell culture.

As used herein, the term “contacting” and its variants, when used in reference to any set of components, includes any process whereby the components to be contacted are mixed into same mixture (for example, are added into the same compartment or solution), and does not necessarily require actual physical contact between the recited components. The recited components can be contacted in any order or any combination (or subcombination), and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting a template with a reaction mixture” includes any or all of the following situations: (i) the template is contacted with a first component of the reaction mixture to create a mixture; then other components of the reaction mixture are added in any order or combination to the mixture; and (ii) the reaction mixture is fully formed prior to mixture with the template.

The term “mixture” as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not addressable. To be specific, an array of surface-bound oligonucleotides, as is commonly known in the art and described below, is not a mixture of surface-bound oligonucleotides because the species of surface-bound oligonucleotides are spatially distinct and the array is addressable.

By “diagnosis” or “evaluation” of a cancer (e.g., a lung cancer or melanoma) refers to a diagnosis of a cancer, a diagnosis of a stage of the cancer, a diagnosis of a type or classification of the cancer, a diagnosis or detection of a recurrence of the cancer, a diagnosis or detection of a regression of the cancer, a prognosis of the cancer, or an evaluation of the response of the cancer to a surgical or non-surgical therapy.

Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other diagnosing and/or staging methods known in the medical art for a particular disease or disorder, e.g., lung cancer or melanoma.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 20” may indicate a range of 18 to 22, and “about 1” may mean from 0.9-1.1. Other meanings of “about” may be apparent from the context, such as rounding off, so, for example “about 1” may also mean from 0.5 to 1.4.

Preferred embodiments are illustrated herein, but those skilled in the art will appreciate that other components and conditions in addition to those illustrated may be used in the methods described herein.

### Example

In this example, the method described above was used for enriching or detecting mutations in EGFR, KRAS, and BRAF from samples of late-stage lung cancer patients. Briefly, blood samples were obtained from nine late-stage lung cancer patients with consent. cfDNA was extracted from the plasma (Samples A-I) using a QIAamp Circulating Nucleic Acid kit (Qiagen) by standard techniques known in the art. Wild-type genomic DNA (Promega) or mutant genomic DNA (Horizon) (i.e. EGFRdel19 or KRASG12V etc.) was sonicated to 200 bp and used as controls.

The cfDNA or sonicated genomic control DNA was ligated with an adapter containing UID and Illumina P7 sequence, using the NEBNext Ultra II DNA Library Prep Kit (NEB). A first PCR using GSP-Illumina P5 and Illumina P7 was performed in the presence (Table 2) or absence of blockers (Table 1). A second PCR using barcode-containing Illumina P5 and P7 primers completed library construction. Libraries were sequenced and bioinformatic analysis using UID information was performed to determine mutation frequency (Table 1) or digitally remove false positives (Tables 1 and 2).

TABLE 1

| Hotspots | | Mutations | Mutation |
|---|---|---|---|
| tested | Sample | Identified | Frequency |
| EGFRe19 | A | EGFRdel19 | 8.8% |
| EGFRT790 | B | none | |
| EGFRL858 | C | EGFRL858R | 2.2% |
| KRASG12 | D | none | |
| KRASQ61 | E | KRASG12C | 0.4% |
| BRAFV600 | F | EGFRT790M | 0.05% |
| | | EGFRL858R | 0.3% |
| | G | EGFRL858R | 0.4% |
| | H | none | |
| | I | none | |
| | WT | none | |
| | control | | |
| | 0.1% | EGFRdel19 | 0.10% |
| | control | EGFRT790M | 0.21% |
| | | EGFRL858R | 0.34% |
| | | KRASG12V | 0.17% |
| | | KRASQ61H | 0.21% |
| | | BRAFV600E | 1.1% |

As shown in Table 1, gene-specific amplification of cfDNA from the nine patients (samples A-I) or sheared genomic DNA (0.1% control) and digital cleanup using tag sequence on the adapter allowed for successful quantification of mutation frequency and removal of false positives. For 0.1% control, the sensitivity and specificity were 93% and 100%, respectively (31 experiments).

TABLE 2

| | | After blocker | Digital |
|---|---|---|---|
| Hotspot | Input | enrichment | Cleanup of WT |
| KRASG12V | 0.10% | 9.25% | 0% |
| BRAFV600E | 0.71% | 46.63% | 0% |
| EGFRL858R | 0.09% | 6.83% | 0% |
| EGFRT790M | 0.13% | 6.17% | 0% |
| EGFRdel19 | 0.08% | 0.60% | 0% |

As shown in Table 2, five different low-frequency mutations were significantly enriched in a single reaction using the blocker approach. For example, the KRASG12V mutation was enriched from 0.1% to 9.25%. Digital cleanup removed false-positives in the wild type samples.

The above results indicate that the method is very sensitive. Mutations with frequencies as low as 0.05% in patients could be successfully identified. Also, over 90% of mutations at 0.1% frequency could be detected successfully. False positives were low due to the UID-based digital cleanup. Blockers significantly enriched low-abundance mutations so that sequencing depth and therefore cost can be decreased.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1. A method for exponential amplification of one or more double-stranded target nucleic acid molecules, comprising,
(a) ligating to each double-stranded target nucleic acid molecule an adapter to produce an end-linked double-stranded nucleic acid molecule, said adapter comprising (i) a paired region and (ii) an unpaired region;
(b) providing (i) an adapter primer that is complementary to a primer binding site in the complement of the unpaired region and (ii) a target-specific primer that is complementary to a binding site in the target nucleic acid molecule; and
(c) amplifying the end-linked double-stranded nucleic acid molecule in an amplification reaction comprising the adapter primer and the target-specific primer to produce a first amplified molecule.

2. The method of claim 1, wherein the unpaired region is a loop, a 5′ and/or 3′ overhang, or a bubble.

3. The method of claim 2, wherein the unpaired region is a loop.

4. The method of claim 3, wherein the loop contains a uracil and the method comprises cleaving the loop by uracil DNA glycosylase (UDG) before the amplifying step.

5. The method of claim 1, wherein the target-specific primer contains a tag sequence at the 5′ end.

6. The method of claim 1, the amplification reaction comprises a first blocker comprising a first sequence that (i) is matched or complementary to the wild-type allele in the target nucleic acid molecule and (ii) is capable of being extended by a DNA polymerase.

7. The method of claim 6, the amplification reaction further comprising a second blocker having a second sequence that is matched or complementary to the complement of the wild-type allele.

8. The method of claim 7, wherein the first or second blocker contains one or more modified nucleic acids or linkages.

9. The method of claim 8, wherein the first or second blocker has a modified nucleic acid or linkage at the 3′ end.

10. The method of claim 8, wherein said modified nucleotides or linkages comprise PNA, LNA, a 2′-O-Methyl nucleic acid, a 2′-O-Alkyl nucleic acid, a 2′-fluoro nucleic acid, a phosphorothioate linkage, and any combination thereof.

11. The method of claim 6, wherein the first or second blocker does not overlap with either the adaptor primer or the target-specific primer.

12. The method of claim 1, wherein the target nucleic acid molecule is a ctDNA.

13-16. (canceled)

17. The method of claim 1, wherein the target nucleic acid molecule spans a region encoding EGFR T790M, EGFR L858R, BRAF V600E, BRAF V600K, BRAF V600D, BRAF V600G, BRAF V600A, or BRAF V600R or KRASG12V.

18. A method of obtaining the sequence of one or more double-stranded target nucleic acid molecules, comprising,
obtaining a first amplified molecule produced according to the method of claim 1,
amplifying the first amplified molecule in a second amplification reaction comprising a pair of primers, each primer having a barcode sequence, to generate a set of second amplified molecules, and
sequencing the second amplified molecules.

19. A method for evaluating a subject having cancer, comprising
obtaining a biological sample from the subject; and
performing an assay to determine the presence or absence of one or more target nucleic acid molecules in the biological sample according to the method of claim 1.

20. The method of claim 19, wherein the biological sample is serum, plasma, whole blood, saliva, or sputum.

21. The method of claim 19, further comprising determining or recommending a treatment course of action based on the presence of said one or more target nucleic acid molecules.

22. The method of claim 21, further comprising a step of administering said treatment when said one or more target nucleic acid molecules are present.

23. The method of claim 1, wherein the amplification reaction is a multiplex amplification reaction.

24. A kit for amplification of a target nucleic acid molecule, comprising
(a) an adapter comprising (i) a paired region and (ii) an unpaired region;
(b) an adapter primer that is complementary to a primer binding site in the complement of the unpaired region, and
(c) a target-specific primer that is complementary to a binding site in the target nucleic acid molecule.

25-31. (canceled)
